(19) United States
(12) Patent Application Publication
(10) Pub. No.: US 2025/0195282 A1
(43) Pub. Date: Jun. 19, 2025

(54) QUICK-DRYING ABSORPTION CORE

(71) Applicant: MEGA SOFT (CHINA) CO., LTD., Quanzhou (CN)

(72) Inventor: Hanhe CHEN, Hongkong (CN)

(21) Appl. No.: 19/066,405

(22) Filed: Feb. 28, 2025

Related U.S. Application Data
(63) Continuation of application No. PCT/CN2023/114003, filed on Aug. 21, 2023.

(30) Foreign Application Priority Data
Aug. 30, 2022 (CN) 202211056278.1

(51) Int. Cl. A61F 13/15; A61F 13/45; A61F 13/53

(52) U.S. Cl. CPC A61F 13/15203; A61F 13/15699; A61F 13/45; A61F 13/53; A61F 2013/15463; A61F 2013/4587; A61F 2013/530481

(57) ABSTRACT

This invention relates to the field of disposable sanitary products, particularly focusing on a highly efficient quick-drying absorption core. The absorption core includes an absorbent core body, which includes upper nonwoven fabric layer, a fluffy nonwoven fabric layer, and a lower nonwoven fabric layer. Super absorbent polymer is positioned on the upper and lower surfaces of the fluffy nonwoven fabric layer, with the polymer being partially embedded within the pores of the fluffy nonwoven fabric layer. The density of the super absorbent polymer on the fluffy nonwoven fabric layer decreases in a stepwise manner from the longitudinal center towards both ends. The upper nonwoven layer is bonded to the upper surface of the fluffy nonwoven layer, while the lower nonwoven layer is bonded to the lower surface of the fluffy nonwoven layer. The absorbent core body is pressed with two guiding channels running along its longitudinal center.

### CROSS-REFERENCE TO RELATED APPLICATIONS

This application is a continuation of International Patent Application No. PCT/CN2023/114003 with a filing date of Aug. 21, 2023, which claims priority to Chinese Patent Application No. 202211056278.1 with a filing date Aug. 30, 2022. The content of the aforementioned application are incorporated herein by reference.

### FIELD OF THE INVENTION

This invention relates to the field of disposable hygiene products, especially to a quick-drying absorption core.

### BACKGROUND OF THE INVENTION

The utilization of absorbent cores extends extensively to hygiene products such as diapers and sanitary napkins. Conventional absorbent cores are composed of a blend of cellulose fibers and super absorbent polymer. These are enveloped in nonwoven fabric to form a block-like structure. However, this type of absorbent core is prone not only to agglomeration and fragmentation during usage but also exhibits considerable thickness and inadequate breathability, thereby diminishing user comfort.

Presently, a widely employed absorbent core in the market features a design involving a double-layer or triple-layer nonwoven fabric arrangement, incorporating a middle layer infused with super absorbent polymer. These hydrogel materials are dispersed onto the upper surface of one layer of nonwoven fabric, followed by lamination with another layer through adhesive spraying. While this variant of absorbent core boasts reduced thickness, the challenge lies in securing the super absorbent polymer effectively, as they are susceptible to displacement or leakage, severely compromising absorbency efficiency.

### SUMMARY OF THE INVENTION

Consequently, in response to the aforementioned issues, this invention presents a high-efficiency, quick-drying absorption core.

To realize the aforementioned objectives, the following technical approach has been adopted.

A quick-drying absorption core includes an absorbent core body. This absorbent core body is longitudinally extended, defining the vertical direction, and laterally extended, defining the horizontal direction. The absorbent core body includes an upper nonwoven fabric layer, a fluffy nonwoven fabric layer, and a lower nonwoven fabric layer. The upper and lower surfaces of the fluffy nonwoven fabric layer are provided with super absorbent polymer. Part of these materials is embedded within the pores of the fluffy nonwoven fabric layer, with the density of super absorbent polymer atop the fluffy nonwoven fabric layer diminishing in a stepwise manner from the longitudinal midpoint towards two ends of the fluffy nonwoven fabric layer. The upper nonwoven fabric layer is adhesively laminated onto the upper surface of the fluffy nonwoven fabric layer, while the lower nonwoven fabric layer is adhesively laminated onto the lower surface of the fluffy nonwoven fabric layer. The lateral width of the lower nonwoven fabric layer is greater by 8 mm to 25 mm than the lateral width of the fluffy nonwoven fabric layer. The lateral sides of the lower nonwoven fabric layer are folded upward and inward, adhering to the upper surface of the upper nonwoven fabric layer, and securing to the upper nonwoven fabric layer and the fluffy nonwoven fabric layer through heat pressing or ultrasonic welding. The longitudinal ends of the upper nonwoven fabric layer is securely joined to the fluffy nonwoven fabric layer and the lower nonwoven fabric layer through heat pressing or ultrasonic welding. Two guidance channels are pressed into the longitudinal center of the absorbent core body, running in parallel along the vertical direction. Heat pressing or ultrasonic welding secures the upper nonwoven fabric layer, the fluffy nonwoven fabric layer, and the lower nonwoven fabric layer at longitudinal center of the guidance channels. The absorbent capacity of the absorbent core body follows a stepwise increment from one longitudinal end to the other, and then similarly decreases in a stepwise manner.

In some embodiments, the two guidance channels are arc-shaped and symmetrically distribute along the horizontal axis of the absorbent core body.

In some embodiments, no super absorbent polymer are introduced into the region of the guidance channels.

In some embodiments, the ratio of the longitudinal length of the welding area within the guidance channels to the longitudinal length of the guidance channels is within the range of 0.5 to 0.85:1.

In some embodiments, the ratio of the longitudinal length of the guidance channels to the longitudinal length of the absorbent core body is between 0.6 to 0.8:1.

In some embodiments, the lateral width of the upper nonwoven fabric layer is greater by 8 mm to 25 mm than that of the fluffy nonwoven fabric layer. The upper nonwoven fabric layer is folded upward and inward along the lateral sides of the upper nonwoven fabric layer, and is positioned between the folding region of the lower nonwoven fabric layer and the upper nonwoven fabric layer.

By adopting the aforementioned technical approach, this invention yields several beneficial effects as follows.

The introduced quick-drying absorption core achieves an incremental and decremental stepwise absorbency from one longitudinal end to the other through the periodic addition of super absorbent polymer onto the upper and lower surfaces of the fluffy nonwoven fabric layer. This design effectively reduces the polymer hydrogel content at the longitudinal edges of the absorbent core body, thus lowering production costs. Additionally, within the oscillations of the fluffy nonwoven fabric layer during transmission, the super absorbent polymer is partially embedded within its pores, serving to limit and secure them. Consequently, the distribution characteristics of the super absorbent polymer within a single absorbent core body are maintained, concentrating these materials in the central longitudinal area for optimal absorption, thereby enhancing efficiency while reducing production expenses. This arrangement curtails super absorbent polymer leakage, ensuring sustained water-absorbing performance. Furthermore, after adhesive lamination of the fluffy nonwoven fabric layer and the upper nonwoven fabric layer, the pressed guidance channels are formed. Subsequent bonding through heat pressing or ultrasonic welding of the upper nonwoven fabric layer, the fluffy nonwoven fabric layer, and the lower nonwoven fabric layer within the longitudinal center region of the guidance channels ensures the prevention of material displacement during pressing, thereby elevating precision during the process. The welded area on the guidance channels sustains structural integrity of the guidance channels even after absorbing a substantial volume of liquid, ensuring effective liquid guidance and heightened absorption speed. Moreover, through adhesive bonding at both longitudinal ends of the guidance channels, an expansion buffer is provided in cases of excessive swelling of the super absorbent polymer. This separation of the upper nonwoven fabric layer, the lower nonwoven fabric layer, and the fluffy nonwoven fabric layer enables enhanced water absorption in the peripheral region, further augmenting the overall absorbent capacity.

### BRIEF DESCRIPTION OF DRAWINGS

FIG. 1 is a top-view schematic diagram the absorbent core according to one embodiment of the present invention.

FIG. 2 is a cross-sectional schematic diagram of section A-A in FIG. 1.

FIG. 3 is a cross-sectional schematic diagram of section B-B in FIG. 1.

FIG. 4 is a schematic flowchart of the method for preparing the absorbent core according to one embodiment of the present invention.

FIG. 5 is a three-dimensional schematic flowchart of the method for preparing the absorbent core according to one embodiment of the present invention.

FIG. 6 is a distribution diagram of absorbency along the longitudinal length direction of the absorbent core body according to one embodiment of the present invention.

FIG. 7 is a cross-sectional diagram of the feeding roller according to one embodiment of the present invention.

### DETAILED DESCRIPTION OF THE INVENTION

In conjunction with the accompanying drawings and specific embodiments, further explanation of the present invention is provided.

The embodiment of the present invention is as follows.

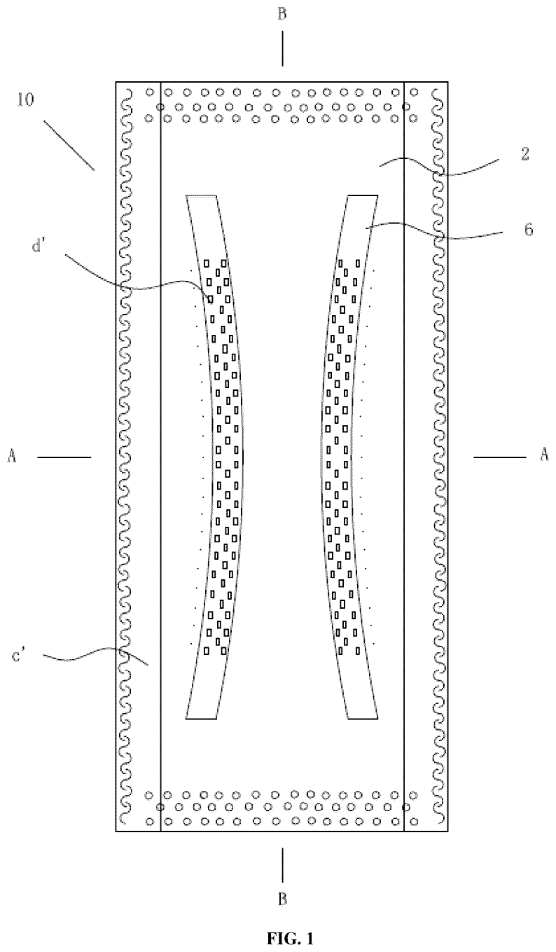

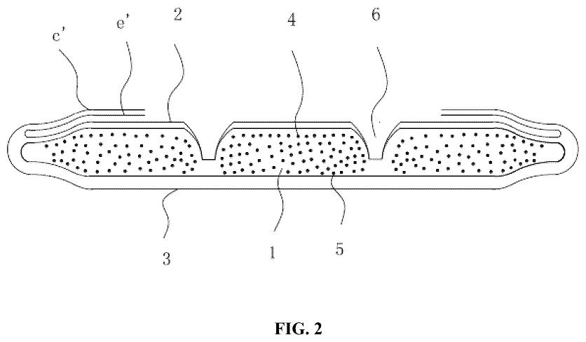

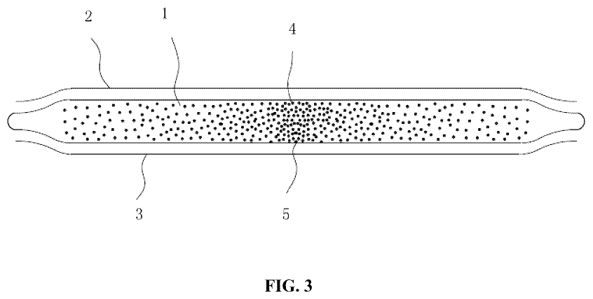

In reference to FIGS. 1, 2, and 3, a quick-drying absorption core includes an absorbent core body denoted as 10. The absorbent core body extends longitudinally and horizontally, defining the longitudinal and horizontal directions, respectively. The absorbent core body 10 includes an upper nonwoven fabric layer 2, a fluffy nonwoven fabric layer 1, and a lower nonwoven fabric layer 3. An upper surface a′ and a lower surface b′ of the fluffy nonwoven fabric layer 1 are each provided with super absorbent polymers 4 and 5, respectively. These super absorbent polymers 4, 5 are periodically introduced and laid out atop the fluffy nonwoven fabric layer 1, with a partial embedding within the pores of the fluffy nonwoven fabric layer 1. The density of these super absorbent polymers 4, 5 on the upper surface a′ of the fluffy nonwoven fabric layer 1 diminishes stepwise from the longitudinal midpoint towards its extremities. The upper nonwoven fabric layer 2 is adhesively bonded onto the upper surface a′ of the fluffy nonwoven fabric layer 1, while the lower nonwoven fabric layer 3 is adhesively bonded onto the lower surface b′ of the fluffy nonwoven fabric layer 1. The lateral width of the lower nonwoven fabric layer 3 is greater by 8 mm to 25 mm than that of the fluffy nonwoven fabric layer 1. Preferably, the lateral width of the lower nonwoven fabric layer 3 is greater by 16 mm than that of the fluffy nonwoven fabric layer 1. The lateral sides c′ of the lower nonwoven fabric layer 3 are folded upward and inward, adhering to the upper surface of the upper nonwoven fabric layer 2 and secured through ultrasonic welding to both the upper nonwoven fabric layer 2 and the fluffy nonwoven fabric layer 1. Similarly, the longitudinal ends of the upper nonwoven fabric layer 2 are ultrasonically welded to the fluffy nonwoven fabric layer 1 and the lower nonwoven fabric layer 3. Two guidance channels 6 are pressed into the longitudinal center of the absorbent core body 10. These guidance channels 6 are arc-shaped, symmetrically distributing along the horizontal axis of the absorbent core body 10. No super absorbent polymers 4, 5 are added into the regions of the guidance channels 6. Ultrasonic welding the upper nonwoven fabric layer, the fluffy nonwoven fabric layer 1, and the lower nonwoven fabric layer 3 at the longitudinal center area of the guidance channels. The absorbent capacity of the core body 10 increases in a stepwise manner from one longitudinal end to the other and then similarly decreases in a stepwise manner. The ratio of the longitudinal length of the welding area d′ within the guidance channels 6 to the longitudinal length of the guidance channels 6 is within the range of 0.5 to 0.85:1, preferably, with an optimal value of 0.75:1. Furthermore, the ratio of the longitudinal length of the guidance channels 6 to the longitudinal length of the absorbent core body 10 is between 0.6 to 0.8:1, with an optimal value of 0.7:1.

Furthermore, the lateral width of the upper nonwoven fabric layer 2 is greater by 8 mm to 25 mm than that of the fluffy nonwoven fabric layer 1. Preferably, the lateral width of the upper nonwoven fabric layer 2 is greater by 14 mm than that of the fluffy nonwoven fabric layer 1. The lateral sides e′ of the upper nonwoven fabric layer 2 are folded upward and inward, positioned between the folding region c′ of the lower nonwoven fabric layer 3 and the upper nonwoven fabric layer 2.

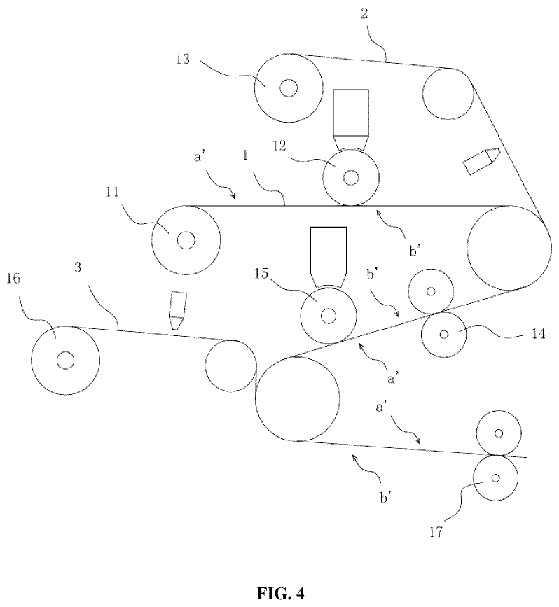

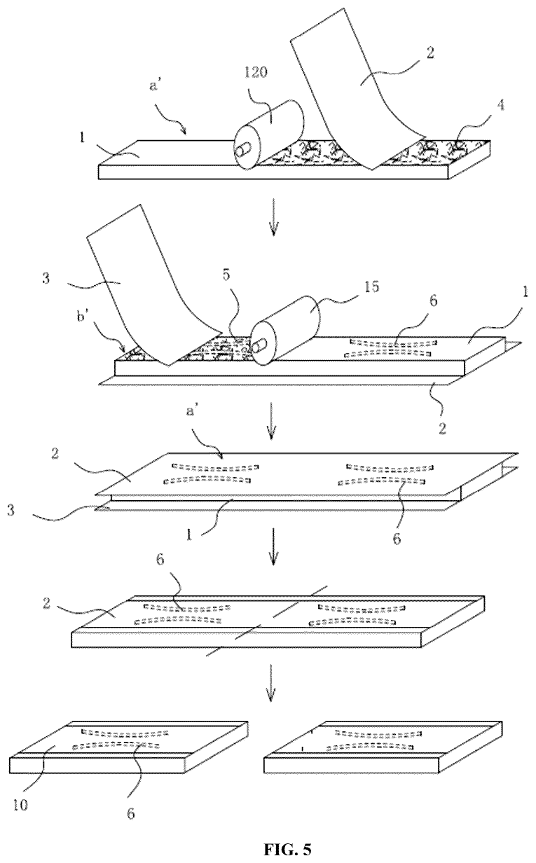

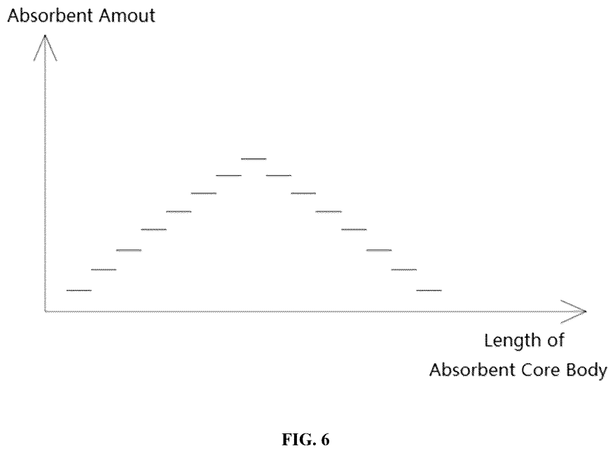

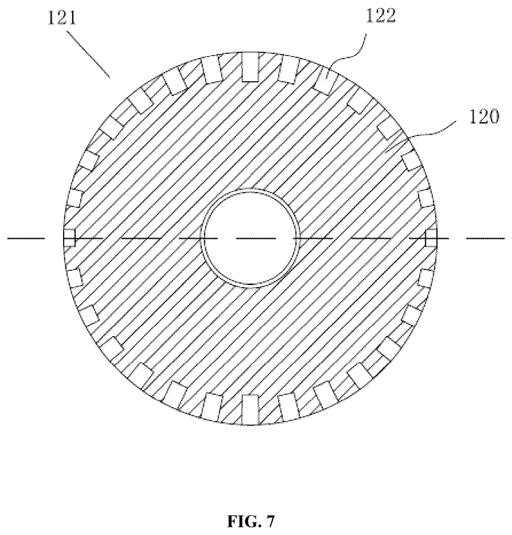

Referring to FIG. 4 to FIG. 7, a method for preparing a quick-absorbing and dry-absorbing core includes the following steps.

1) Preparing the core layer, wherein the core layer includes continuous absorbent units, defining a transportation direction as longitudinal direction, and a lateral across of the core layer as horizontal direction.
a) Unwinding and transporting, by the first unwinding unit 11, the fluffy nonwoven fabric layer 1; the first unwinding unit 11 includes a servo motor connected to a control unit; the start time and rotational speed of the servo motor is determined by the control unit, thereby establishing the starting point and transport speed of the fluffy nonwoven fabric layer 1.
b) Obtaining the starting point and transport speed of the fluffy nonwoven fabric layer 1 according to step a, and utilizing pre-inputted longitudinal lengths of the absorbent units within the control unit to determine the boundaries of each absorbent unit.
c) Obtaining the boundaries of each absorbent unit based on step b, and using the first feeding unit 12 to periodically add super absorbent polymer 4 to the upper surface a′ of the fluffy nonwoven fabric layer 1. The super absorbent polymer 4 is partially embedded within the pores of the fluffy nonwoven fabric layer 1. The first feeding unit 12 includes a feeding roller 120, with the outer surface of the feeding roller 120 having at least one feeding area 121 that matches an absorbent unit. The coverage area of the feeding area 121 is equal to the area of a single absorbent unit. Preferably, the feeding roller 120 has two feeding areas 121. Each feeding area 121 is provided with a material storage trough 122 for storing the super absorbent polymer. This ensures that the boundaries of each absorbent unit correspond to the feeding areas 121 on the feeding roller 120. When the feeding roller 120 rotates for feeding, the super absorbent polymer 4 is accurately fed to the corresponding individual absorbent unit through a single feeding area 121.
d) Unwinding and transporting, by the second unwinding unit 13, the upper nonwoven fabric layer 2. Applying adhesive to the lower surface of the upper nonwoven fabric layer 2, and adhering the upper nonwoven fabric layer 2 to the upper surface a′ of the fluffy nonwoven fabric layer 1.
e) Obtaining the boundaries of each absorbent unit from step b, and utilizing an embossing roller unit 14 to press a guidance channel 6 onto the product formed in step d. This guidance channel 6 is located in the longitudinal center of each absorbent unit.
f) Flipping the product from step e by 180° so that the upper surface a′ of the fluffy nonwoven fabric layer 1 is exchanged with the lower surface b′, and the lower surface b′ of the fluffy nonwoven fabric layer 1 becomes the upper surface.
g) Obtaining the boundaries of each absorbent unit from step b, and using the second feeding unit 15 to periodically add super absorbent polymer 5 to the lower surface b′ of the fluffy nonwoven fabric layer 1. The super absorbent polymer 5 is partially embedded within the pores of the fluffy nonwoven fabric layer 1.
h) Unwinding and transporting, by the third unwinding unit 16, the lower nonwoven fabric layer 3. Applying adhesive to the lower surface of the lower nonwoven fabric layer 3, and adhering the lower nonwoven fabric layer 3 to the lower surface b′ of the fluffy nonwoven fabric layer 1. The lateral width of the lower nonwoven fabric layer 3 is greater by 8 mm to 25 mm than that of the fluffy nonwoven fabric layer 1. Preferably, the lateral width of the lower nonwoven fabric layer 3 is greater by 16 mm than that of the fluffy nonwoven fabric layer 1.
i) Flipping the product from step h by 180° so that the positions of the upper nonwoven fabric layer 2 and the lower nonwoven fabric layer 3 are exchanged.
j) Folding the lateral sides of the lower nonwoven fabric layer 3 upward and inward, attaching it to the upper surface of the upper nonwoven fabric layer 2, and fixing them together through heat pressing or ultrasonic welding.
k) Obtaining the boundaries of each absorbent unit from step b and the position of the guidance channel 6 from step e, and using heat pressing or ultrasonic welding to the upper nonwoven fabric layer 2, the fluffy nonwoven fabric layer 1, and the lower nonwoven fabric layer 3 together at the longitudinal center area of the guidance channel 6 in the of the product from step j.

2) Obtaining the boundaries of each absorbent unit from step b, and using heat pressing or ultrasonic welding to secure the upper nonwoven fabric layer 2, the fluffy nonwoven fabric layer 1, and the lower nonwoven fabric layer 3 on both sides of the absorbent units within the core layer in step 1.

3) Obtaining the boundaries of each absorbent unit based on step b, and using a cutting unit 17 to cut along the boundaries of the absorbent units in the product obtained in step 2. This forms individual absorbent core bodies 10, and the absorbent capacity of each absorbent core body 10 gradually increases and then decreases from one end to the other along the longitudinal direction.

The method of preparation for the quick-drying absorption core involves the periodic incorporation of super absorbent polymer onto the upper and lower surfaces of the fluffy nonwoven fabric layer 1. This results in a graduated increase followed by a decrease in the moisture absorption of the fabricated absorbent core body 10 along its longitudinal axis, creating a stepwise profile. This design effectively reduces the concentration of super absorbent polymer on the longitudinal edges of the absorbent core body 10, thus reducing costs. Furthermore, during the transmission vibrations of the fluffy nonwoven fabric layer 1, the super absorbent polymer becomes partly embedded within the pores of the fabric, thereby securing super absorbent polymer in place. This strategic placement maintains the distribution characteristics of the super absorbent polymer content within individual absorbent core bodies 10. Concentrating the super absorbent polymer in the longitudinal center region of the absorbent core body 10 enhances its priority absorption function, leading to heightened absorption efficiency and decreased production expenses. This method also achieves rapid absorption, while effectively mitigating the risk of super absorbent polymer leakage and preserving optimal moisture absorption performance. By implementing the aforementioned preparation procedure, product enhancements can be seamlessly incorporated into the existing core production line with minimal adjustments, resulting in reduced investment costs. Furthermore, following the adhesive lamination of the fluffy nonwoven fabric layer 1 and upper nonwoven fabric layer 2, the formation of guidance channels 6 is achieved through pressing. Subsequently, through a combination of heat pressing and ultrasonic welding, the upper nonwoven fabric layer 2, fluffy nonwoven fabric layer 1, and lower nonwoven fabric layer 3 are firmly connected within the central longitudinal area of the guidance channels 6. This approach not only prevents material deviations during the pressing of guidance channels 6 but also enhances the precision of the process. In addition, the welded area of the guidance channels 6 maintains its structural integrity even after absorbing significant amounts of liquid, ensuring optimal guidance performance. This enhancement leads to a heightened rate of liquid absorption within the product. Furthermore, the adhesive lamination technique is employed at both ends of the guidance channels 6 to facilitate separation between the upper nonwoven fabric layer 2, lower nonwoven fabric layer 3, and fluffy nonwoven fabric layer 1. This innovative design offers a buffer space to accommodate excessive expansion caused by the super absorbent polymer's substantial swelling. Consequently, this refinement maximizes moisture absorption, further elevating the overall liquid-absorbing capacity.

Specifically, the density of the super absorbent polymer 4 within the absorbent core body 10 diminishes in a stepwise manner from the longitudinal center region to both ends of the absorbent core body 10. Similarly, the density of the super absorbent polymer 5 within the absorbent core body 10 follows a similar stepwise reduction trend from the longitudinal center region to both ends of the absorbent core body 10. Consequently, around the circumference of the feeding area 121 along the lower feeding roller 120, the volume of the material storage trough 122 within the feeding area 121 undergoes a progression of expansion followed by reduction. This cyclic rotation and periodic addition of the super absorbent polymer through the storage trough 122 onto the fluffy nonwoven fabric layer 1 generates a stepwise distribution pattern. This design effectively reduces the equipment investment cost associated with the feeding roller, while the stepwise distribution pattern enhances the precision of material deposition, ensuring a growth in product moisture absorption efficiency.

Alternatively, the density of the super absorbent polymer 4 within the absorbent core body 10 gradually decreases from the longitudinal center region to both ends of the absorbent core body 10, while the density of the super absorbent polymer 5 within the absorbent core body 10 maintains a consistent distribution from the longitudinal center region to both ends. Alternatively, the density of the super absorbent polymer 4 within the absorbent core body 10 maintains a consistent distribution from the longitudinal center region to both ends, whereas the density of the super absorbent polymer 5 follows a stepwise reduction trend from the longitudinal center region to both ends of the absorbent core body 10. All of these variations achieve the aforementioned effects.

It is noted that the absence of periodic addition of the super absorbent polymers 4 and 5 within the region of the guidance channel 6 ensures the unimpeded efficacy of the guidance channel 6, thus enhancing the water absorption efficiency. Moreover, this design provides additional expansion space for the super absorbent polymer, thereby further augmenting the water absorption capacity.

In this embodiment, the ratio of the longitudinal length of the welding area within the guidance channel 6 to the longitudinal length of the guidance channel 6 is in the range of 0.5 to 0.85:1, preferably 0.75:1. Additionally, the ratio of the longitudinal length of the guidance channel 6 to the longitudinal length of the absorbent core body 10 is in the range of 0.6 to 0.8:1, preferably 0.7:1. This arrangement ensures that the guidance channel 6 is distributed along the longitudinal direction of the absorbent core body 10, covering regions with a higher concentration of super absorbent polymer. This facilitates fast absorption during guidance. The configuration of the welding area within the guidance channel 6 optimizes both the absorption efficiency and capacity of the absorbent core body 10.

Furthermore, in step j described above, the lateral width of the upper nonwoven fabric layer 2 is greater than the lateral width of the bulky nonwoven fabric 1 by 8 mm to 25 mm, preferably 14 mm. The lateral sides of the upper nonwoven fabric layer 2 are folded upward and inward in tandem with the lateral sides of the lower nonwoven fabric layer 3, sandwiched between the folding region of the lower nonwoven fabric layer 3 and the upper nonwoven fabric layer 2. This configuration prevents lateral leakage of the super absorbent polymer and establishes side barriers on the surface of the absorbent core body 10, enhancing the prevention of lateral liquid seepage during usage and improving overall effectiveness.

Testing was conducted on the prepared absorbent core body 10, and the test data are as follows:

Quick-absorbing Core Test Data-BIO-L-3

| Water Retention Capacity (g) | | | | | BSF Test | | | | |
|---|---|---|---|---|---|---|---|---|---|
| | Sample | Sample | Sample | | | Sample | Sample | Sample | |
| | 1 | 2 | 3 | Ave. | | 1 | 2 | 3 | Ave. |
| Sample | 20.46 | 20.48 | 20.47 | 20.47 | Sample | 20.53 | 20.58 | 20.66 | 20.59 |
| Weight | | | | | Weight | | | | |
| Saturation | 650.53 | 620.54 | 628.53 | 633.20 | Absorption | 8.31 | 9.56 | 10.03 | 9.30 |
| Capacity (g) | | | | | Time (s) | | | | |
| | | | | | Diffusion | 300 | 280 | 300 | 293.33 |
| | | | | | Length (mm) | | | | |
| | | | | | Rewet | 0.11 | 0.09 | 0.06 | 0.09 |
| | | | | | Amount (g) | | | | |
| Water | 535.54 | 520.52 | 512.53 | 522.86 | Absorption | 3.34 | 3.41 | 3.62 | 3.46 |
| Retention (g) | | | | | Time (s) | | | | |
| | | | | | Diffusion | 340 | 330 | 360 | 343.33 |
| | | | | | Length (mm) | | | | |
| | | | | | Rewet | 0.08 | 0.06 | 0.07 | 0.07 |
| | | | | | Amount (g) | | | | |
| Standard | 0.03 | 0.04 | 0.04 | 0.04 | Absorption | 4.0 | 3.81 | 4.1 | 3.97 |
| Rewet (g) | | | | | Time (s) | | | | |
| | | | | | Diffusion | 380 | 380 | 380 | 380 |
| | | | | | Length (mm) | | | | |
| | | | | | Rewet | 0.13 | 0.12 | 0.12 | 0.12 |
| | | | | | Amount (g) | | | | |

| | |
|---|---|
| Testing Date | Jul. 19, 2022 |

An alternate implementation in this embodiment is as follows.

A method for preparing a quick-drying absorption core includes the following steps.

1) Preparing the core layer, wherein the core layer includes continuous absorbent units, defining a transportation direction as longitudinal direction, and a lateral across of the core layer as horizontal direction.
a) Unwinding and transporting, by the first unwinding unit 11, the fluffy nonwoven fabric layer 1. The first unwinding unit 11 includes a servo motor connected to a control unit. The start time and rotational speed of the servo motor is determined by the control unit, thereby establishing the starting point and transport speed of the fluffy nonwoven fabric layer 1.
b) Obtaining the starting point and transport speed of the fluffy nonwoven fabric layer 1 according to step a, and utilizing pre-inputted longitudinal lengths of the absorbent units within the control unit to determine the boundaries of each absorbent unit.
c) Obtaining the boundaries of each absorbent unit from step b, and using the first feeding unit 12 to periodically add super absorbent polymer 4 to the upper surface a′ of the fluffy nonwoven fabric layer 1. The super absorbent polymer 4 is partially embedded within the pores of the fluffy nonwoven fabric layer 1. The first feeding unit 12 includes a feeding roller 120, with the outer surface of the feeding roller 120 having at least one feeding area 121 that matches an absorbent unit. The coverage area of the feeding area 121 is equal to the area of a single absorbent unit. Preferably, the feeding roller 120 has two feeding areas 121. Each feeding area 121 is provided with a material storage trough 122 for storing the super absorbent polymer. This ensures that the boundaries of each absorbent unit correspond to the feeding areas 121 on the feeding roller 120. When the feeding roller 120 is rotated for feeding, the super absorbent polymer 4 is accurately fed to the corresponding individual absorbent unit through a single feeding area 121.
d) Unwinding and transporting, by the second unwinding unit 13, the upper nonwoven fabric layer 2. Applying adhesive to the lower surface of the upper nonwoven fabric layer 2, and adhering the upper nonwoven fabric layer 2 to the upper surface a′ of the fluffy nonwoven fabric layer 1.
e) Obtaining the boundaries of each absorbent unit from step b, and utilizing an embossing roller unit 14 to press a guidance channel 6 onto the product formed in step d. This guidance channel 6 is located in the longitudinal center of each absorbent unit.
f) Flipping the product from step e by 180° so that the upper surface a′ of the fluffy nonwoven fabric layer 1 is exchanged with the lower surface b′, and therefore the lower surface b′ of the fluffy nonwoven fabric layer 1 becomes the upper surface.
g) Obtaining the boundaries of each absorbent unit from step b, and using the second feeding unit 15 to periodically add super absorbent polymer 5 to the lower surface b′ of the fluffy nonwoven fabric layer 1. The super absorbent polymer 5 is partially embedded within the pores of the fluffy nonwoven fabric layer 1.
h) Unwinding and transporting, by the third unwinding unit 16, the lower nonwoven fabric layer 3. Applying adhesive to the lower surface of the lower nonwoven fabric layer 3, and adhering the lower nonwoven fabric layer 3 to the lower surface b′ of the fluffy nonwoven fabric layer 1. The lateral width of the lower nonwoven fabric layer 3 is greater by 8 mm to 25 mm than that of the fluffy nonwoven fabric layer 1. Preferably, the lateral width of the lower nonwoven fabric layer 3 is greater by 16 mm than that of the fluffy nonwoven fabric layer 1.
i) Folding the lateral sides of the lower nonwoven fabric layer 3 upward and inward, attaching it to the upper surface of the upper nonwoven fabric layer 2, and fixing them together through heat pressing or ultrasonic welding.
j) Obtaining the boundaries of each absorbent unit from step b and the position of the guidance channel 6 from step e, and using heat pressing or ultrasonic welding to the upper nonwoven fabric layer 2, the fluffy nonwoven fabric layer 1, and the lower nonwoven fabric layer 3 together at the longitudinal center area of the guidance channel 6 in the of the product from step i.

2) Obtaining the boundaries of each absorbent unit from step b, and using heat pressing or ultrasonic welding to secure the upper nonwoven fabric layer 2, the fluffy nonwoven fabric layer 1, and the lower nonwoven fabric layer 3 on both sides of the absorbent units within the core layer in step 1.

3) Obtaining the boundaries of each absorbent unit based on step b, and using a cutting unit 17 to cut along the boundaries of the absorbent units in the product obtained in step 2. This forms individual absorbent core bodies 10, and the absorbent capacity of each absorbent core body 10 gradually increases and then decreases from one end to the other along the longitudinal direction.

While the preferred embodiments have been presented and described in detail in conjunction with the present invention, those skilled in the art should understand that various changes can be made to the present invention without departing from the spirit and scope of the claims attached hereto. Such changes, whether in form or detail, remain within the scope of protection of the present invention as defined by the claims.

What is claimed is:

1. A quick-drying absorption core, characterized by comprising an absorbent core body; the absorbent core body is longitudinally extended, defining a vertical direction, and laterally extended, defining a horizontal direction; the absorbent core body comprises an upper nonwoven fabric layer, a fluffy nonwoven fabric layer, and a lower nonwoven fabric layer; an upper surface and a lower surface of the fluffy nonwoven fabric layer are provided with super absorbent polymer; part of the super absorbent polymer is embedded within the pores of the fluffy nonwoven fabric layer, with the density of super absorbent polymer atop the fluffy nonwoven fabric layer diminishing in a stepwise manner from the longitudinal midpoint towards two ends of the fluffy nonwoven fabric layer; the upper nonwoven fabric layer is adhesively laminated onto an upper surface of the fluffy nonwoven fabric layer, while the lower nonwoven fabric layer is adhesively laminated onto a lower surface of the fluffy nonwoven fabric layer; the lateral width of the lower nonwoven fabric layer is greater by 8 mm to 25 mm than the lateral width of the fluffy nonwoven fabric layer; the lateral sides of the lower nonwoven fabric layer are folded upward and inward, adhering to the upper surface of the upper nonwoven fabric layer, and securing to the upper nonwoven fabric layer and the fluffy nonwoven fabric layer through heat pressing or ultrasonic welding; the longitudinal ends of the upper nonwoven fabric layer is securely joined to the fluffy nonwoven fabric layer and the lower nonwoven fabric layer through heat pressing or ultrasonic welding; two guidance channels are pressed into the longitudinal center of the absorbent core body, running in parallel along the vertical direction; the upper nonwoven fabric layer, the fluffy nonwoven fabric layer, and the lower nonwoven fabric layer are jointed securely at longitudinal center of the guidance channels by heat pressing or ultrasonic welding secures; the absorbent capacity of the absorbent core body follows a stepwise increment from one longitudinal end to the other, and then similarly decreases in a stepwise manner.

2. The absorbent core according to claim 1, characterized in that the two guidance channels are arc-shaped and symmetrically distribute along the horizontal axis of the absorbent core body.

3. The absorbent core according to claim 1, characterized in that no super absorbent polymer is introduced into the region of the guidance channels.

4. The absorbent core according to claim 1, 2 or 3, characterized in that the ratio of the longitudinal length of the welding area within the guidance channels to the longitudinal length of the guidance channels is within the range of 0.5 to 0.85:1.

5. The absorbent core according to claim 4, characterized in that the ratio of the longitudinal length of the guidance channels to the longitudinal length of the absorbent core body is between 0.6 to 0.8:1.

6. The absorbent core according to claim 4, characterized in that the lateral width of the upper nonwoven fabric layer is greater by 8 mm to 25 mm than that of the fluffy nonwoven fabric layer; the upper nonwoven fabric layer is folded upward and inward along the lateral sides of the upper nonwoven fabric layer, and is positioned between the folding region of the lower nonwoven fabric layer and the upper nonwoven fabric layer.
